(19) United States
(12) Patent Application Publication
(10) Pub. No.: US 2014/0128681 A1
(43) Pub. Date: May 8, 2014

(54) INTUBATION GUIDE ASSEMBLY

(71) Applicant: COOK MEDICAL TECHNOLOGIES LLC, Bloomington, IN (US)

(72) Inventor: Amie L. Fordinal, Houston, TX (US)

(73) Assignee: Cook Medical Technologies LLC, Bloomington, IN (US)

(21) Appl. No.: 13/671,935

(22) Filed: Nov. 8, 2012

(51) Int. Cl. A61B 1/267; A61M 16/04

(52) U.S. Cl. CPC A61B 1/267; A61M 16/0488
USPC 600/194

(57) ABSTRACT

An intubation guide assembly includes a laryngoscope having a handle and a blade. The blade has a first portion engaged with the handle, and a second portion configured for insertion into the oral cavity of a patient. The handle and blade are configured for slidably receiving a guide member. The guide member is dimensioned such that a proximal portion is extendable beyond the handle for manipulation by an operator, and a distal portion is extendable into the trachea of the patient when the blade is inserted into the oral cavity. Upon withdrawal of the laryngoscope, a tubular airway member may be advanced over the guide member such that the distal end of the airway member extends into the trachea to provide ventilation to the patient.

### BACKGROUND OF THE INVENTION

1. Technical Field

The present disclosure relates generally to airway management devices. More particularly, the disclosure relates to an intubation guide assembly useful for positioning an endotracheal tube in the airway of a patient.

2. Background Information

Ventilation is a physiologic process which supplies oxygen to the body and removes carbon dioxide, a gaseous waste product. Ventilation is provided by the rhythmic back and forth motion of air in the trachea, caused by the rhythmic contraction and relaxation of the diaphragm. In seriously ill or injured patients unable to breathe adequately on their own, ventilation can be assisted by inserting an endotracheal tube through the oral or nasal cavity of a patient, a process often referred to as endotracheal intubation. An endotracheal tube is a balloon-tipped single or double-lumen catheter that is open at both ends. One end extends outside of the patient and is engaged with a mechanical ventilator for supplying a ventilation fluid. The other end extends between the vocal cords and into the trachea of the patient.

Proper placement of the endotracheal tube typically requires the use of a guide instrument, such as a laryngoscope, to provide a degree of visualization of the internal anatomy of the patient. The laryngoscope may include a curved blade-like structure that is inserted into the pharynx. The blade-like structure elevates the epiglottis to provide a view of the vocal cords and the glottis, and provides a pathway for the end of the endotracheal tube to be manually directed past the vocal cords, and into the trachea. A handle engaged with the blade extends outside the throat to facilitate manipulation by the medical professional. During the intubation procedure, the professional typically grasps the handle of the laryngoscope with one hand, and controls the position of the endotracheal tube with the other hand.

With the patient lying on his or her back, the laryngoscope is typically inserted into the mouth on the right side, and then moved to the left side to move the tongue out of the line of sight. The blade is then lifted in an upward and forward motion to elevate the epiglottis such that the line of sight to the glottis is achieved. During this intubation process, the presence of a second, and sometimes even a third, person is generally required in order to manipulate the patient's head and jaw into alignment to enable optimal visualization of the vocal cords, and to assist with insertion of the endotracheal tube. Since both the laryngoscope and the endotracheal tube must be inserted into a small space in the vicinity of the vocal cords, the intubation procedure typically requires a high degree of experience and care on the part of the intubation team in order to ensure proper visualization and placement of the endotracheal tube, and to avoid damage to the vocal cords and other anatomical structures during the process of inserting the tube.

It would be desirable to provide a guide assembly to enable the physician to manage insertion of the laryngoscope blade and endotracheal tube into the mouth with a single hand, thereby freeing the second hand to perform other actions, such as manipulating the position of the jaw, or holding open the mouth of the patient. This action can reduce the number of medical personnel involved in the intubation procedure, and reduce the time required for intubating the patient.

### BRIEF SUMMARY

The present invention addresses the shortcomings of the prior art. In one form thereof, the invention comprises an intubation guide assembly comprising a laryngoscope having a handle and a blade member. The blade member has a first portion operatively engaged with the handle and a second portion configured for insertion into the oral cavity of a patient. The handle and blade member are configured for receiving a guide member therealong. A guide member is slidably received along the handle and the blade member. The guide member is dimensioned such that a proximal portion is extendable beyond the handle for manipulation by an operator, and a distal portion is extendable into the trachea of the patient when the blade member is inserted into the oral cavity.

In another form thereof, the invention comprises an intubation system. The intubation system includes a laryngoscope comprising a handle and a blade member. The blade member has a first portion engaged with the handle and a second portion configured for insertion into the oral cavity of a patient. The blade member includes at least one retaining member therealong. A guide member is slidably received along the handle and the blade member. The guide member is dimensioned such that a proximal portion is extendable beyond the handle for manipulation by an operator and a distal portion is extendable into the trachea of the patient when the blade member is inserted into the oral cavity. A tubular airway member is receivable over the guide member. The tubular airway member has a first end engageable with a source of ventilation, and a second end extendable into the trachea of the patient.

In still another form, the invention comprises a method for intubating a patient. A laryngoscope is initially positioned for insertion into the oral cavity of the patient. The laryngoscope comprises a handle and a blade member. The blade member has a first end engaged with the handle, and has a second end. Each of the handle and blade member is configured for slidably receiving a guide member therealong. The second end of the blade member is inserted into the oral cavity, and the blade member is maneuvered therein such that a line of sight is provided to an interior space of the oral cavity. A guide member is slidably advanced along the handle and blade member such that a distal end of the guide member extends into the trachea of the patient. The laryngoscope is withdrawn, while maintaining the guide member distal end in the trachea.

### BRIEF DESCRIPTION OF THE DRAWINGS

FIG. 1 is a view of an intubation guide assembly according to one version of the present invention;

FIG. 2 is an enlarged view of a clip member of the intubation guide assembly of FIG. 1;

FIG. 3 is an enlarged view of a blade member of the intubation guide assembly of FIG. 1;

FIG. 4 is a view of an intubation guide assembly according to another version of the present invention;

FIG. 5 is a bottom view of the intubation guide assembly of FIG. 4;

FIG. 6 is a view showing oral insertion of the intubation guide assembly, and advancement of the guide member into the trachea;

FIG. 7 illustrates the guide member inserted into the trachea following removal of the laryngoscope; and

FIG. 8 illustrates insertion of an endotracheal tube over a guide member.

### DESCRIPTION OF THE PREFERRED EMBODIMENTS

For purposes of promoting an understanding of the present invention, reference will now be made to the embodiments illustrated in the drawings, and specific language will be used to describe the same. It should nevertheless be understood that no limitation of the scope of the invention is thereby intended, such alterations and further modifications in the illustrated device, and such further applications of the principles of the invention as illustrated therein being contemplated as would normally occur to one skilled in the art to which the invention relates.

In the following discussion, the terms “proximal” and “distal” will be used to describe the opposing axial ends of the intubation guide assembly, as well as the axial ends of various component features. The term “proximal” is used in its conventional sense to refer to the end of the assembly (or component) that is closest to the medical professional during use of the assembly. The term “distal” is used in its conventional sense to refer to the end of the assembly (or component) that is initially inserted into the patient, or that is closest to the patient during use.

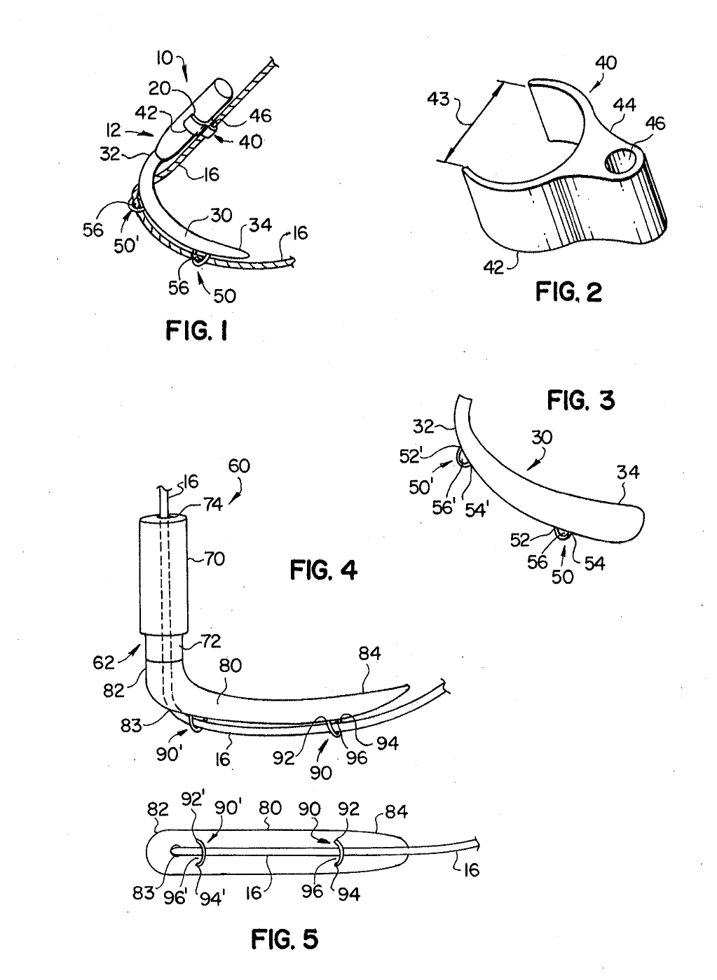

FIG. 1 is a perspective view of one example of an intubation guide assembly 10. Intubation guide assembly 10 comprises a laryngoscope 12 and a guide member 16 extending along the laryngoscope. Laryngoscope 12 includes a handle 20 and a blade member 30. Handle 20 may be configured in any manner that enables the medical professional to securely grasp and control the laryngoscope. Typically, handle 20 comprises a generally cylindrical barrel. If desired, the outer grasping surface of the barrel may be knurled, dimpled, grooved, etc., to enhance grippability.

Blade member 30 is configured for insertion into the oral cavity of the patient in well-known manner. A proximal end 32 of the blade member may be permanently or detachably engaged with the distal end of the handle by any well-known means, e.g., by a screw connection, a mechanical engagement via mating structures on respective adjacent parts, adhesion, and the like. Blade member 30 is dimensioned in well-known fashion to facilitate insertion into the throat area of the patient. Blade member 30 may have any surface commonly utilized for such purposes in known laryngoscopes. Examples of suitable blade surfaces include a curved surface, such as the Macintosh blade, and a straight surface, such as the Miller blade. Although the Macintosh blade and the Miller blade are well-known blades for use in laryngoscopy, those skilled in the art will appreciate that other blade structures will also be suitable for use in the intubation guide assembly as described herein.

Laryngoscopes are now well known in the intubation art. Laryngoscopes may be configured to provide a direct line of sight to the larynx, and may include a light source positioned along the distal end 34 of the blade. Laryngoscopes having a light source may be provided with a power source, such as one or more batteries, positioned along the laryngoscope, e.g., along or interior of the handle. Alternatively, the laryngoscope may be provided with a visualization mechanism, such as a fiberoptic wire, camera, or scope, which is extendable into the trachea from the blade. Such visualization mechanisms provide indirect views of the larynx on a monitor. Those skilled in the art will appreciate that many, if not most, laryngoscopes known in the art will be suitable for use in the intubation guide assembly described herein.

In addition to the laryngoscope 12, intubation guide assembly 10 includes a guide member 16. As shown above, guide member 16 extends along the length of the laryngoscope, and further extends a distance proximal to, and distal to, the respective ends of the laryngoscope. Guide member 16 may comprise a catheter having a small outer diameter (e.g., about 1-6 mm) or a wire guide, bougie, or like device of similar dimension. The guide member should have a length such that it extends in the proximal direction beyond the proximal end of the handle to enable grasping by the physician, and extends in the distal direction beyond the distal end of the blade and into the trachea to provide a pathway for the endotracheal tube, as described herein.

One example of a suitable guide member is the Cook Airway Exchange Catheter, available from Cook Medical Incorporated of Bloomington, Ind. Cook Airway Exchange Catheters are available in various lengths and diameters, and are readily receivable in a passageway through an endotracheal tube. One benefit to the use of such catheters is that adapters are available for engaging the proximal end of the catheter with a source of oxygen. In this manner, oxygen can be provided to the patient through the catheter once the catheter is fed through the vocal cords. Such adapters are commercially available from Cook Medical Incorporated, and marketed as RAPI-FIT® adapters. Although catheters of other dimensions are available for use in a particular application, the preferred Airway Exchange Catheters described above have an outer diameter between about 8 and 19 French (2.7 and 6.3 mm), a length between about 45 and 83 cm, and an inner diameter between about 1.6 and 3.4 mm. Those skilled in the art can readily select a catheter having suitable dimensions for a particular application.

In the example of FIG. 1, a retaining member is provided along the length of handle 20. The retaining member may have any configuration suitable for maintaining a position of guide member 16 in sliding relationship with laryngoscope handle 20. One example of a suitable retaining member is clip member 40. FIG. 2 is an enlarged view of one example of a clip member 40 prior to engagement with the laryngoscope handle 20. In this example, clip member 40 comprises a generally ring-like body 42 configured to fit, or clip, over the generally cylindrical barrel of handle 20. Clip member 40 preferably is formed from a flexible material, and may include a void 43 along a part of the circumference of ring-like member 42. Void 43 enables the flexible clip member 40 to be readily clipped or otherwise fitted onto the barrel of handle 20, as shown in FIG. 1. The presence of void 43 also allows the flexible clip member to self-adjust in order to accommodate different handle sizes. In the example shown, clip member 40 also includes an extended portion 44 having an aperture 46 extending therethrough. Aperture 46 is dimensioned to slidably receive and retain guide member 16 (FIG. 1).

Although clip member 40 is one example of a retaining member for slidably engaging the guide member and the laryngoscope handle, those skilled in that art will appreciate that other members capable of slidably retaining the guide member in this fashion may be substituted, and are considered within the scope of the invention. The retaining member need not necessarily be formed from a flexible material, and need not necessarily be detachable. Rather, the retaining member may be formed of a rigid or semi-rigid composition, such as a rigid plastic, with or without the void. In this arrangement, the retaining member may be affixed or otherwise engaged with the handle 20 by any suitable means, such as adhesion or a friction fit. As a still further alternative, the retaining member may be integrally formed with the handle by known means, e.g., injection molding.

One or more additional retaining members may be affixed or otherwise engaged with the laryngoscope. In one example, retaining members 50, 50′ are affixed to blade member 30, e.g., as shown in FIGS. 1 and 3. In the non-limiting example shown, retaining members 50, 50′ are affixed to the underside of the blade. Retaining members 50, 50′ may comprise any member capable of engagement with the blade member and having a passageway therethrough to slidably receive the guide member. In the non-limiting example shown, retaining members 50, 50′ comprise a wire or like member having dual ends 52, 52′, and 54, 54′, respectively. Ends 52, 52′ and 54, 54′ are securely affixed to the blade underside in a manner to define a passageway 56, 56′ for guide member 16. As with aperture 46, passageway 56, 56′ is dimensioned to slidably receive and retain the guide member (FIG. 1).

Those skilled in the art will recognize that it is desirable to dimension the retaining members such that the guide member is slidably receivable therethrough, and yet the retaining member has as low a profile as possible along the blade underside. The relative dimension of the retaining members with reference to the blade will typically be smaller than shown in the figures, wherein the dimensions of the retaining members have been exaggerated for ease of illustration.

In one variation, one of the wire ends need not necessarily be affixed to the blade underside. In this variation, the non-affixed wire end (for example, ends 54, 54′) will be substantially configured in the manner of the wire ends shown in the figures, other than the fact that it is not mechanically affixed to the blade underside. This example provides an element of freedom to allow wires of different diameters to pass along the blade, as the position of the retaining members is adjustable vis-à-vis the blade underside to permit larger diameter guide members to pass along the blade underside. Those skilled in the art will appreciate that in this variation wires 50, 50′ will preferably be formed of a relatively stiff composition (e.g., stainless steel or nitinol) having a strength sufficient to maintain as low a profile as possible of the wire against the blade underside.

Another version of an intubation guide assembly 60 is shown in FIG. 4. The intubation guide assembly 60 described and shown in this example includes many features in common with the previous example. For example, the intubation guide assembly 60 includes a laryngoscope 62, having a handle 70 and a blade member 80. As with handle 20 of the previous example, handle 70 may be configured in any manner that enables the medical professional to securely grasp and control the laryngoscope. Handle 70 may include any of the surface modifications described above for enhancing grippability. In the example shown, handle 70 comprises a generally cylindrical barrel.

Unlike the handle of the previous example, handle 70 includes a passageway 74 extending axially through the handle. In this example, passageway 74 extends the length of handle 70, and is dimensioned for receiving guide member 16.

The blade member 80 has a proximal end 82 and a distal end 84. As with the previous example, proximal end 82 of the blade member may be permanently or detachably engaged with an end of the handle by conventional means. For such engagement, handle 70 may include a downwardly directed extension 72 or like structure that receives (or alternatively, is received in) a corresponding structure of blade proximal end 82. As best shown in the bottom view of FIG. 5, proximal end 82 of blade member 80 includes an aperture 83 in communication with passageway 74. Guide member 16 may be advanced through passageway 74 and aperture 83 such that it extends along the underside of the blade 80.

One or more retaining members may be affixed to the underside of the blade in the same manner as retaining members 50, 50′ of the previous example. In this example, retaining members 90, 90′ are provided along the underside of blade member 80. Retaining members 90, 90′ may comprise any wire or like member having dual ends 92, 92′ and 94, 94′ that are securely affixed to the blade underside in a manner to define a passageway 96, 96′ for guide member 16. Passageways 96, 96′ are dimensioned to slidably receive and retain guide member 16 in the same manner as passageways 56, 56′ of the previous version.

The following example illustrates one use of an intubation guide assembly as described and shown herein. In the example, intubation guide assembly 60 is utilized to secure an airway of a patient, in order to facilitate insertion of an endotracheal tube into the airway.

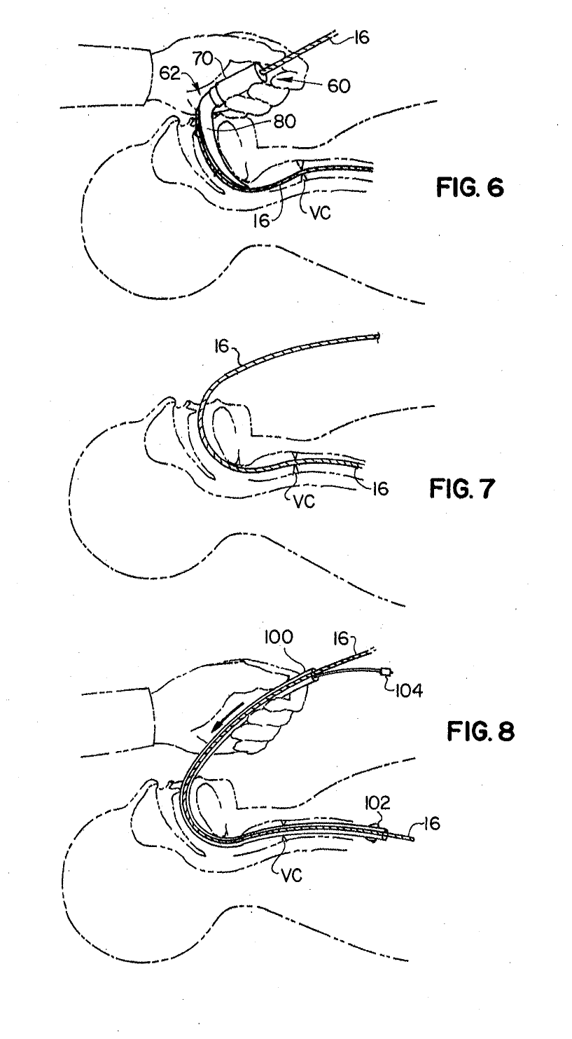

FIG. 6 illustrates use of intubation guide assembly 60 during an intubation process. As described previously, laryngoscope 62 includes handle 70 and blade member 80. Handle 70 includes longitudinal passageway 74 that receives guide member 16. Those skilled in the art understand that there are many types of laryngoscopes and like intubation guide devices in use in the medical field, and that minor modifications to the procedure described herein may be required for use with some such devices. It is believed that such modification is well within the ability of those skilled in the art.

In this example, the patient is reclining on his/her back. The medical professional initially manipulates the jaw of the patient in well-known fashion for entry of the laryngoscope blade. The blade is then inserted and maneuvered in conventional fashion such that a line of sight to the vocal cords and the opening between them (the glottis) is achieved. Typically, this involves inserting the blade at the right side of the mouth, and moving the blade toward the left side to move the tongue away from the sight line. Such actions are well-known in the art and further details of the insertion procedure are not necessary for an understanding of the process described herein,

Once a line of sight to the vocal cords and glottis has been achieved, the medical professional then feeds the guide member through the laryngoscope and into the glottis. In the example of FIG. 6, this is carried out by thumb feeding the slidable guide member 16 through laryngoscope handle 70. Under direct view as provided by the laryngoscope, guide member 16 is then advanced along the underside of laryngoscope blade member 80 through this opening between the vocal cords VC, until the distal end of the guide member extends into the trachea.

The laryngoscope is then withdrawn in a proximal direction over the guide member, leaving the guide member in position. This is shown in FIG. 7, wherein the distal end of guide member 16 is shown extending beyond the glottis into the trachea,

The ventilation tube, such as a conventional endotracheal tube, is then inserted over the guide member. Endotracheal tube 100 may be provided with a conventional inflatable cuff 102 at the distal end, and a pilot balloon 104 at the proximal end for inflating the cuff. Endotracheal tube 100 having the cuff 102 and pilot balloon 104 is only one example of a suitable endotracheal tube, and those skilled in the art will appreciate that other known endotracheal tubes, as well as conventional breathing tubes of other types, may be substituted.

As shown in FIG. 8, endotracheal guide 100 is initially advanced over guide member 16 along the oral cavity, and thereafter is further advanced beyond the glottis and into the trachea. Guide member 16 may then be withdrawn out the proximal end of the endotracheal tube. At this time, the pilot balloon may be activated to inflate the cuff, and the proximal end of the endotracheal tube can be connected to a conventional ventilator (not shown), all in well-known fashion.

It is therefore intended that the foregoing detailed description be regarded as illustrative rather than limiting, and that it be understood that it is the following claims, including all equivalents, that are intended to define the spirit and scope of this invention.

1. An intubation guide assembly, comprising:
a laryngoscope comprising a handle and a blade member, said handle comprising an elongated gripping member having a retaining member affixed thereto along a length thereof, said blade member having a first portion operatively engaged with said handle and a second portion configured for insertion into the oral cavity of a patient, said blade member comprising a retaining member along a length thereof, each of said handle and blade member retaining members being configured for slidably receiving a guide member therealong; and
a guide member slidably received along said handle and said blade member retaining members, said guide member dimensioned such that a proximal portion is extendable beyond said handle for manipulation by an operator and a distal portion is extendable into the trachea of the patient when the blade member is inserted into said oral cavity.

2. (canceled)

3. The intubation guide assembly of claim 1, wherein said handle retaining member and said blade retaining member each comprise an aperture for slidably receiving said guide member.

4. The intubation guide assembly of claim 3, wherein said handle retaining member comprises a clip member configured to be removably affixed to said elongated gripping member, said clip member including an extended portion having said aperture extending therethrough.

5. (canceled)

6. The intubation guide assembly of claim 1, wherein said blade member comprises a second retaining member along said blade member length for receiving said guide member, said second retaining member positioned distal of said first retaining member along an underside of said blade member length.

7. The intubation guide assembly of claim 1, wherein said guide member comprises an elongated catheter.

8. The intubation assembly of claim 1, wherein said handle comprises an elongated gripping member having a channel extending along a length thereof, said channel dimensioned for slidably receiving said guide member.

9. The intubation guide assembly of claim 1, wherein said guide member comprises one of a wire guide and an elongated catheter.

10. An intubation system, comprising:
a laryngoscope comprising a handle and a blade member, said handle comprising a gripping member having a retaining member affixed to said handle along a length thereof, said blade member having a first portion engaged with said handle and having a second portion configured for insertion into the oral cavity of a patient, said blade member including at least one retaining member for receiving a guide member;
a guide member slidably received along said retaining members of said handle and said blade member, said guide member dimensioned such that a proximal portion is extendable beyond said handle for manipulation by an operator and a distal portion is extendable into the trachea of the patient when the blade member is inserted into said oral cavity; and
a tubular airway member receivable over said guide member, said tubular airway member having a first end engageable with a source of ventilation and a second end extendable into the trachea of the patient.

11. (canceled)

12. The intubation system of claim 10, wherein said handle retaining member comprises a clip member configured to be removably received along said elongated gripping member, said clip member including an extended portion having an aperture extending therethrough for receiving said guide member.

13. The intubation system of claim 10, wherein said handle comprises an elongated gripping member having a channel extending longitudinally therethrough, said channel dimensioned for slidably receiving said guide member.

14. The intubation system of claim 10, wherein said at least one retaining member of said blade member comprises a plurality of retaining members disposed along an underside of said blade member.

15. The intubation system of claim 10, wherein said guide member comprises an elongated catheter.

16. A method for intubating a patient, comprising:
positioning a laryngoscope for insertion into the oral cavity of the patient, the laryngoscope comprising a handle and a blade member, the handle comprising an elongated gripping member having a retaining member affixed thereto along a length thereof, said blade member having a first end engaged with said handle and having a second end, the blade member comprising at least one retaining member along a length thereof, each of said handle and blade member configured for slidably receiving a guide member therealong;
inserting the second end of the blade member into the oral cavity, and maneuvering said blade member therein such that a line of sight is provided to an interior space of said oral cavity;
slidably advancing a guide member along said handle and blade member retaining members such that a distal end of said guide member extends into the trachea of the patient; and
withdrawing said laryngoscope, while maintaining said guide member distal end in the trachea.

17. The method of claim 16, further comprising:
advancing a tubular airway member over said guide member such that a distal end of said tubular airway member extends into the trachea; and withdrawing said guide member from the trachea.

18. The method of claim 16, wherein said handle retaining member comprises a removable clip having an aperture for receiving said guide member.

19. The method of claim 16, wherein said handle comprises an elongated gripping member having a channel extending longitudinally therethrough, said channel dimensioned for slidably receiving said guide member.

20. The method of claim 16, wherein said at least one blade member retaining member comprises a plurality of retaining members disposed along an underside of said blade member for slidably receiving said guide member.
